(19) United States
(12) Patent Application Publication
(10) Pub. No.: US 2016/0341799 A1
(43) Pub. Date: Nov. 24, 2016

(54) BATTERY-POWERED DEVICE WITH A SELF-TEST MODE

(71) Applicant: Roost, Inc., Sunnyvale, CA (US)

(72) Inventors: Roel Peeters, San Carlos, CA (US); James Blackwell, Los Gatos, CA (US); James Machiorletti, Antioch, CA (US)

(21) Appl. No.: 14/717,361

(22) Filed: May 20, 2015

(51) Int. Cl. G01R 31/40; G01N 33/00

(52) U.S. Cl. CPC G01R 31/40; G01N 33/0009

(57) ABSTRACT

A battery-powered device includes internal processing capability and battery control capability. Using the battery control capability, in a test mode, an internal processor controls the battery such that the battery provides a lower than normal voltage to some powered portion of the battery-powered device and sensors detect results of the lowering of the voltage. In some battery-powered devices, the lowering of a source voltage for circuits of the device triggers an audio signal and that audio signal is detected by sensors coupled to the internal processor.

### FIELD

The present disclosure relates generally to battery-powered devices that include internal processing capability.

### BACKGROUND

Many devices that did not traditionally have communications capabilities are being replaced by updated devices that do have native communications capabilities. Many of those devices are battery powered. Some of those battery-powered devices can run out of battery energy and then provide an audio signal indicative of a low battery state. This can be at inconvenient times, as might occur with smoke detectors that indicate their low battery state in during the night.

### SUMMARY

A battery-powered device includes internal processing capability and battery control capability. Using the battery control capability, in a test mode, an internal processor controls the battery such that the battery provides a lower than normal voltage to some powered portion of the battery-powered device and sensors detect results of the lowering of the voltage. In some battery-powered devices, the lowering of a source voltage for circuits of the device triggers an audio signal and that audio signal is detected by sensors coupled to the internal processor.

The following detailed description together with the accompanying drawings will provide a better understanding of the nature and advantages of the present invention.

### BRIEF DESCRIPTION OF THE DRAWINGS

FIG. 1 illustrates a novel battery-based device with integrated audio sensing.

FIG. 2 is a schematic diagram of circuits that might be used for a self-test.

FIG. 3 is a schematic diagram of a batter and a voltage sensor.

FIG. 4 is a flowchart of a process for testing a response of circuit.

FIG. 5 is an illustration of circuits described here being used within a conventional smoke detector housing.

FIG. 6 illustrates an example of a battery module.

FIG. 7 illustrates the battery module with its component housings separated.

FIG. 8 illustrates the battery module with its component housings separated with the connection points more visible.

FIG. 9 illustrates a connector as might be used between different boards in different housings.

### DETAILED DESCRIPTION

For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

In embodiments of devices explained herein, a battery-powered device includes a processor that can monitor sensors and control battery voltage seen by components of the battery-powered device.

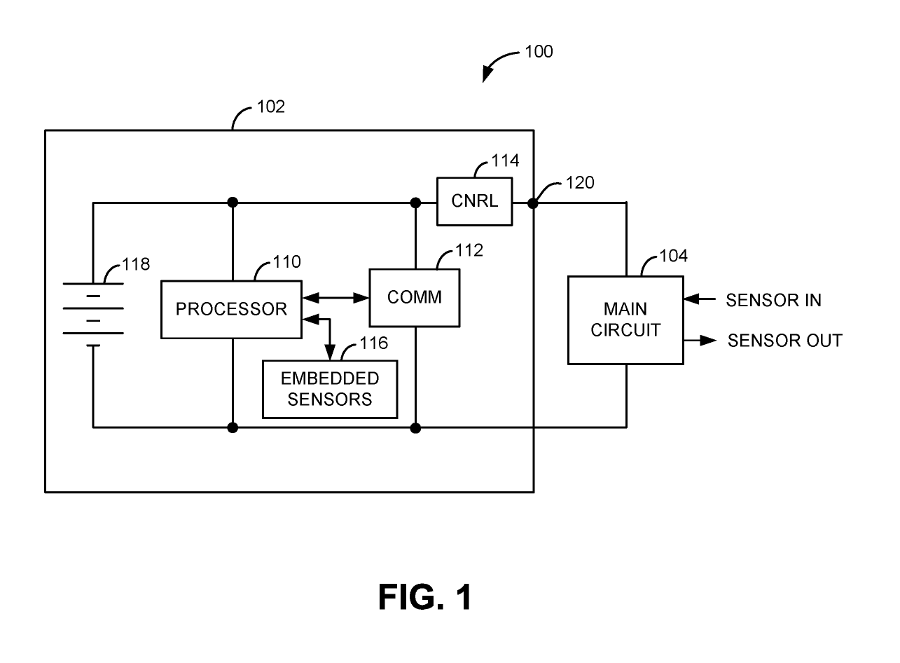

FIG. 1 is a schematic diagram showing various components as might be used. As shown there, a device 100 includes a battery module 102 and a main circuit 104. As shown, main circuit 104 is able to receive sensor input and provide signals as outputs. In a specific use, main circuit 104 is a conventional smoke detector circuit and battery module 102 is a module that can supply the power necessary for operations of that conventional smoke detector circuit. However, other main circuits are also contemplated, such as carbon monoxide alarms and water alarms.

Battery module 102, as shown, includes a processor 110, a communications submodule 112, a battery module output controller 114, embedded sensors 116, and a battery 118. Processor 110 is usable to manage operations of battery module 102, according to processor instructions readable by processor 110. Communications submodule 112 might be a wireless communication circuit and being coupled to processor 110, this would allow processor 110 to send and receive data external to device 100. In some implementations, communications submodule 112 is integrated into processor 110. Battery module output controller 114 is able to control what output supply voltage appears at the supply output node 120 of battery module 102, and might also control what current is drawn from that supply output. Embedded sensors 116 might include an audio sensor, a temperature sensor, and a battery voltage sensor. Battery 118 might be a 9V power source.

In other variations, processor 110 is replaced with a simpler control circuit or some other form of processing circuit. Processor 110 can be a microprocessor, microcontroller, or a system on a chip, as appropriate. Battery module output controller 114 might be replaced with a simple active switch or other supply voltage controller.

Battery module 102 might be integrated into a housing, or multiple interconnected housings such that battery module 102 would fit into a chamber sized to accept a conventional battery. For example, embedded sensors 116 and battery 118 might be integrated into one housing that can be attached, electrically and mechanically, to another housing containing processor 110, communications submodule 112, and battery module output controller 114.

Processor 110 might have a sleep mode and an awake mode, wherein power consumption is reduced in the sleep mode relative to the awake mode. In the awake mode, processor 110 might execute tests of various elements of device 100. For example, processor 110 can execute a test of a communication link by sending a message and waiting for a response, then when a response is received, checking the response for correctness. Processor 110 can also test some operations of main circuit 104, as explained in more detail below.

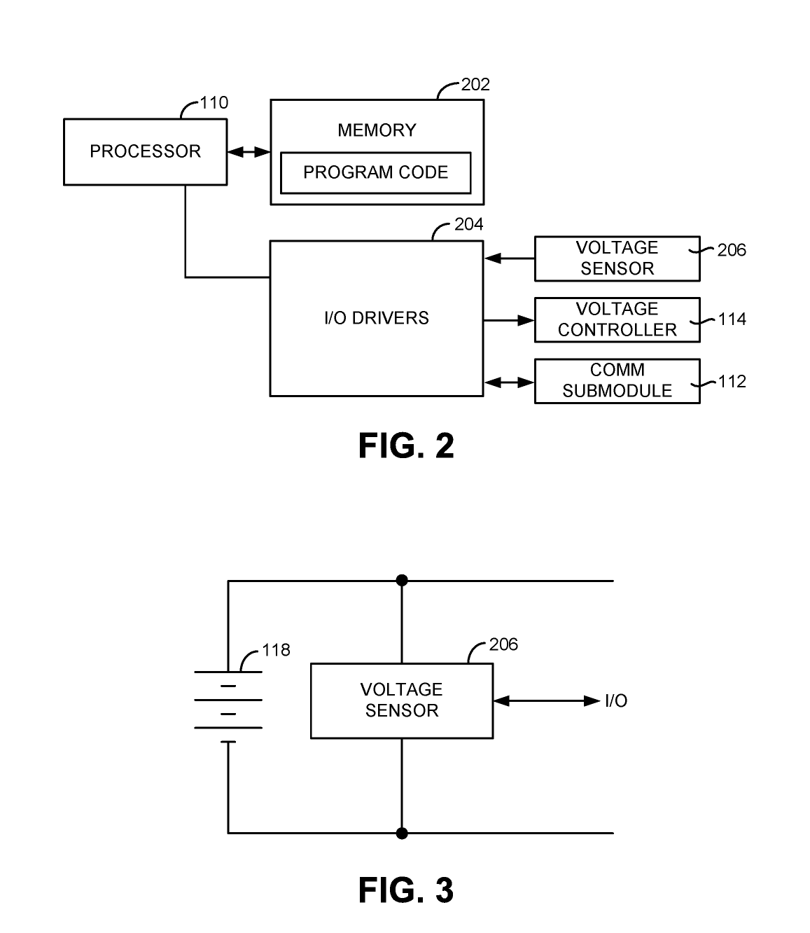

FIG. 2 is a schematic of battery module 102 and various components of battery module 102. For clarity, power supply and ground connections for those components that need them are not shown in FIG. 2, but it should be understood that components needing power to operate are provided power when operational, perhaps via wires, circuit board traces, connections, or electrical powering means not shown.

As shown in FIG. 2, battery module 102 might include processor 110, and memory 202. Memory 202 might be volatile, non-volatile, or both, and might contain program code memory in which program code is stored that processor 110 executes when so instructed. Battery module 102 also might include, as needed, input/output (“I/O”) drivers 204 that are controllable by processor 110 and handle inputs from sensors and the like and outputs to other components, such as a voltage sensor 206, battery module output controller 114, and communications submodule 112.

In an example operation using components shown in FIG. 2, processor 110 might execute program code read from memory 202 that instructs processor 110 to cause I/O drivers 204 to instruct or control battery module output controller 114 to lower or raise a supply voltage that would appear at supply output node 120 for testing a test device or testing a circuit under test.

FIG. 3 illustrates an arrangement of battery 118 and voltage sensor 206. Together, these elements would allow for processor 110 to determine what voltage battery 118 is capable of supplying. In combination with controlling battery module output controller 114, processor 110 could further determine what voltage battery 118 is capable of supplying when loaded by main circuit 104 and when unloaded by main circuit 104. This might provide for more detailed monitoring of a battery module.

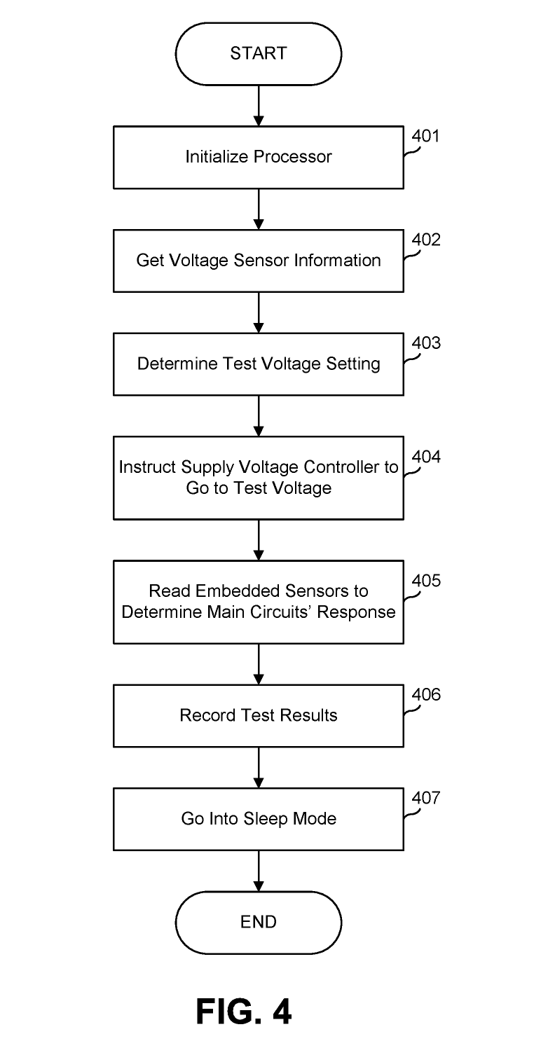

FIG. 4 is a flowchart of a process for testing a response of main circuit 104 to a voltage drop. In that process, at step 401, the processor (processor 110 or the like) is initialized. This could be waking from a sleep mode, or finishing up other tasks and then being instructed to begin a testing process. At step 402, the processor reads information from voltage sensor 206 or similar, to obtain a baseline voltage available from battery 118. At step 403, the processor determines how far to drop the voltage at a supply output node. This might be determined by reading prestored variables or performing a calculation. In other variations, there are pre-specified voltage levels, a higher one (for normal operation) and a lower one (for testing, to trigger a chirp or other signal of low battery. At step 404, the processor instructs or controls a battery module output controller (such as battery module output controller 114) so as to achieve the targeted voltage at the supply output node.

At step 405, the processor reads responses from embedded sensors to determine the tested main circuit's response, and then at step 406, the processor records the response, and if so programmed, at step 407, goes into a sleep mode.

Where the main circuit is a smoke detector circuit and the processor is part of a battery module, the processor might cause the voltage at a supply output node to drop to simulate a smoke detector battery wearing out. When the voltage drop is considered by the smoke detector to be indicative of the battery wearing out, the smoke detector can be expected to emit a chirping sound, such as a short chirp every few minutes, or at some other interval. Embedded sensors such as an audio sensor would sense the emission of the short chirps and the processor can get information from the embedded sensors. This would close the loop, wherein the processor causes a supply voltage to go down and the processor would detect that the smoke detector circuit under test chirped in response. The chirp might not happen immediately, so the device should stay in this test mode for an amount of time that is sufficient to detect a chirp regardless of timing, such as staying in the test mode for two minutes, to catch the case where a device only chirps once per minute. Once a chirp is detected, the alarm has been successfully tested. If after the pre-specified time, if no chirp has been detected, either the alarm is faulty or the battery is not in the alarm any longer. Both of these negative outcomes would flag a notification to the owner.

The processor might be programmed to periodically run this test once per week, once per month, or on some other schedule, and then transmit the results external to the device under test. This would allow for advance message notifications notifying a resident, owner, manager, property owner, or other interested party about a main circuit failure without having to be present to test a device. Since the test for a smoke detector would result in a physical sound indicative of a battery replacement need, the tests might be done when it is known that no occupants are present or specifically in response to an occupant's request to test. In the latter case, presumably the occupant would not be surprised or alarmed if a smoke detector chirped in response to that person's request to test. Occupancy might be determined by other sensors, such as occupancy sensors present in a programmable thermostat.

In operation, a device might perform or be involved with a process to be described. The test of the device is preferably coordinated using a computer, a smartphone app, or other user interface. A user or maintainer of the device might set a schedule in which to test the device. Where the device might be disruptive during tests, the schedule could include times where people are not expected to be around the device. The user might set this information using a smartphone app, which would then communicate it to the device. The communication to the device might be indirect, with the app communicating to a server or cloud storage and the device obtaining the communicated settings by accessing the server or cloud storage. At the scheduled time, the device wakes up any necessary components, performs the self-test and reports the result back to the server, which can then send a notification to the subscribed users indicating the outcome of the test. For the test itself, the alarm device under test activates its sounder for a brief period of time and the alarm device listens for the sound to determine if it is still working. The alarm could check both the envelope and the amplitude of the signal.

A specific method of testing would be to (1) lower an output voltage from a controllable battery to below a threshold required to set the device to its low battery alert mode, (2) wait and detect the device low battery chirp audio signal using the same transducer used to detect alarm signals, and (3) once the test has been completed successfully, increase the output voltage of the battery. The test fails if the battery doesn't detect the low battery chirp within some pre-defined period. The audio sensor portion of embedded sensors 116 might be a piezoelectric transducer.

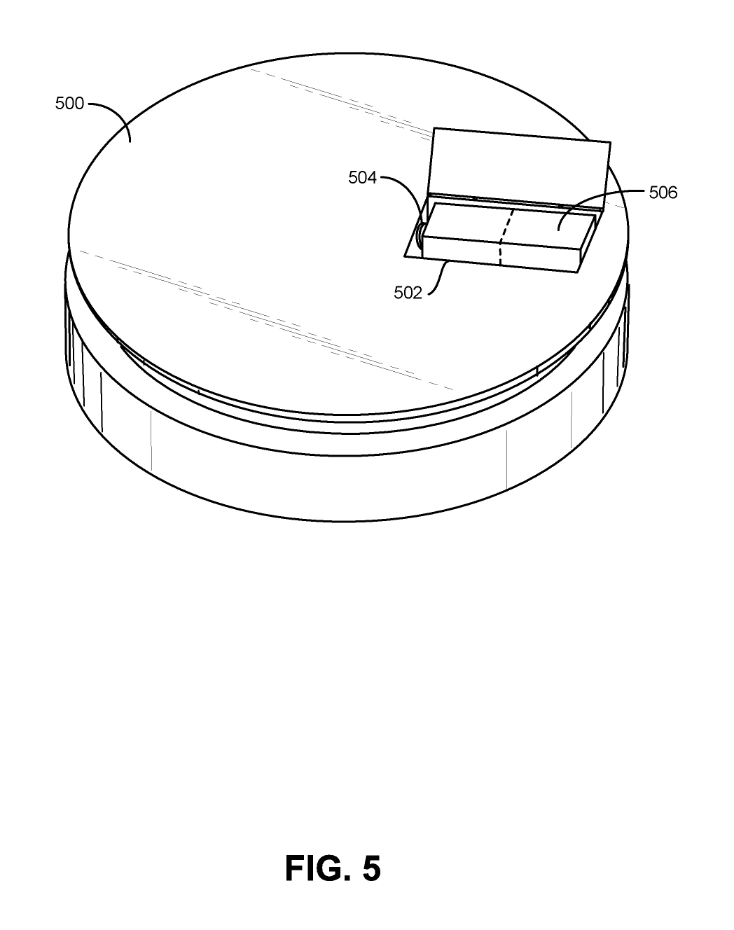

FIG. 5 illustrates how the circuits described above might be used within a conventional smoke detector housing. As illustrated there, smoke detector 500 has a battery compartment that might otherwise house a conventional 9V battery. In its place is battery module 102 in one or more parts.

Device 100 might also be used in other applications, such as a carbon monoxide detector or other alarm condition signaling system. The device might be used with various battery form factors, such as 9V, AA, AAA, ½ AA, N, or other form factors. Using the above concepts, users of devices and sellers of such devices or sellers of combined battery/communications elements might have the systems set up so that tests can be automatically run. The testing feature can also be used to detect the presence of a battery in a device. The lack of a chirp could mean either the battery has been removed or the device is faulty.

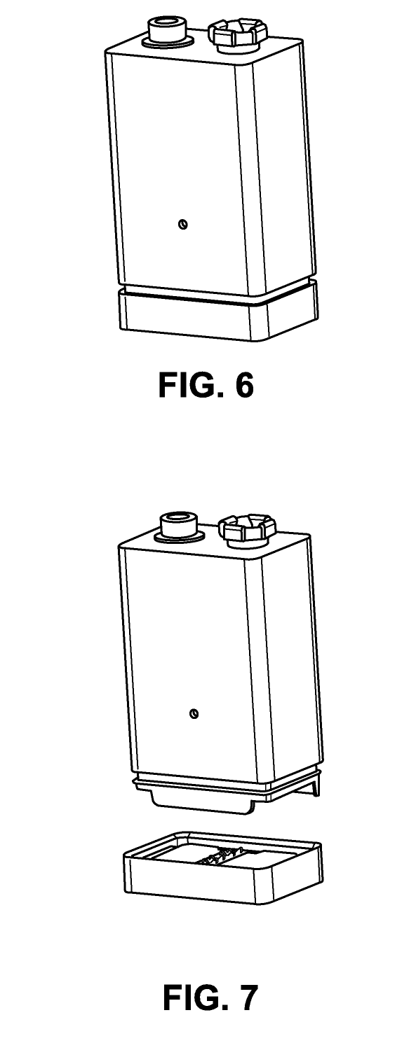

FIG. 6 illustrates an example of a battery module.

FIG. 7 illustrates the battery module with its component housings separated.

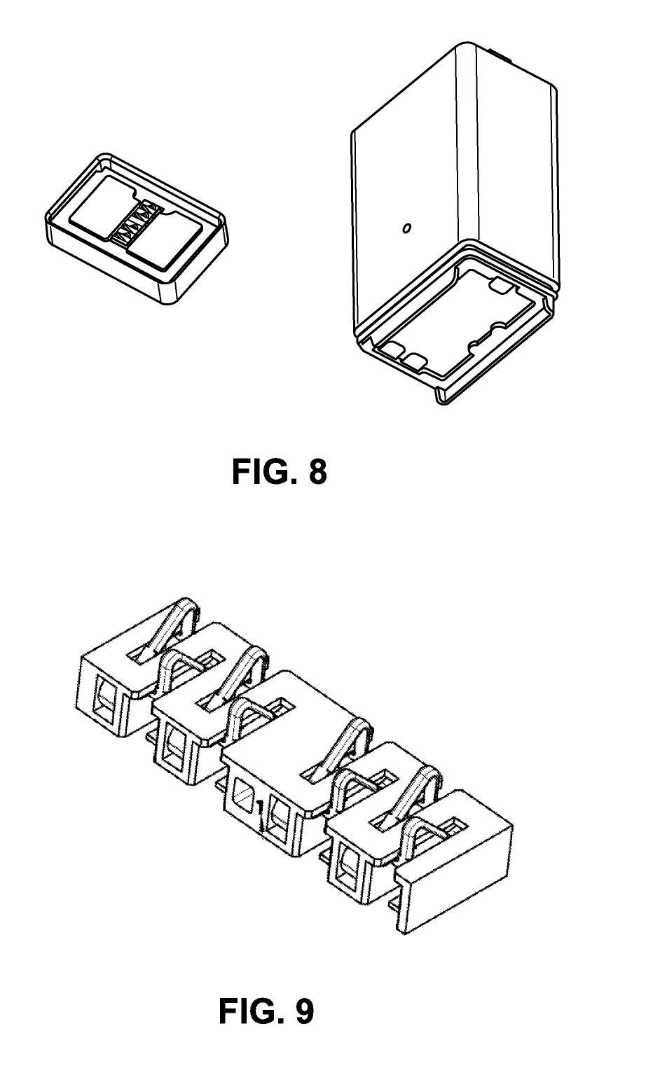

FIG. 8 illustrates the battery module with its component housings separated with the connection points more visible.

FIG. 9 illustrates a connector as might be used between the different boards in different housings (Wi-Fi and processor in one, battery and embedded sensors in the other). In a specific embodiment, the connector is a connector made by Molex with a part number of 78732-8021. In one embodiment, the signals that pass between the boards are (1) I2C Data, (2) I2C Clock, (3) Ground, (4) Audio Sensor, (5) Sensor Interrupt/GPIO, (6) Battery Voltage Control (1-8.2 v, 0-6.9 v), (7) Battery Output Enable, and (8) VBat.

The connector is on the wireless module PCB and mates with gold pads on the battery PCB and held in place by the mechanical connection between the battery pack and wireless module. The connector allows for the separation of the power and sensor functions from the wireless and control functions. By using the same mechanical connection and the defined electrical connections, the wireless module can be connected to different sensor and power packs to create different wireless sensor products.

A battery-powered device with a self-test mode has now been described. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Further embodiments can be envisioned to one of ordinary skill in the art after reading this disclosure. In other embodiments, combinations or sub-combinations of the above-disclosed invention can be advantageously made. The example arrangements of components are shown for purposes of illustration and it should be understood that combinations, additions, re-arrangements, and the like are contemplated in alternative embodiments of the present invention. Thus, while the invention has been described with respect to exemplary embodiments, one skilled in the art will recognize that numerous modifications are possible.

For example, the processes described herein may be implemented using hardware components, software components, and/or any combination thereof. The specification and drawings are, accordingly, to be regarded in an illustrative rather than a restrictive sense. It will, however, be evident that various modifications and changes may be made thereunto without departing from the broader spirit and scope of the invention as set forth in the claims and that the invention is intended to cover all modifications and equivalents within the scope of the following claims.

All references, including publications, patent applications, and patents, cited herein are hereby incorporated by reference to the same extent as if each reference were individually and specifically indicated to be incorporated by reference and were set forth in its entirety herein.

What is claimed is:

1. A test device comprising:
a battery that supplies a supply voltage;
a processing circuit;
a supply voltage controller, controllable by the processing circuit, for controlling an output supply voltage that is powered by the supply voltage;
a program code memory; and
program code for testing a circuit under test that is powered by the output supply voltage, wherein testing includes lowering the output supply voltage and detecting a response from the circuit under test.

2. The test device of claim 1, further comprising a housing sized to fit into a battery compartment.

3. The test device of claim 2, wherein the battery compartment is a battery compartment of a smoke detector.
